(19) United States
(12) Patent Application Publication
(10) Pub. No.: US 2022/0252608 A1
(43) Pub. Date: Aug. 11, 2022

(54) URINARY EXOSOME BIOMARKER FOR DIAGNOSING ANTIBODY-MEDIATED REJECTION AFTER KIDNEY TRANSPLANTATION OR PREDICTING PROGNOSIS OF PATIENT AFTER KIDNEY TRANSPLANTATION

(71) Applicants: UNIVERSITY OF ULSAN FOUNDATION FOR INDUSTRY COOPERATION, Ulsan (KR); THE ASAN FOUNDATION, Seoul (KR)

(72) Inventor: Sung SHIN, Seoul (KR)

(73) Assignees: UNIVERSITY OF ULSAN FOUNDATION FOR INDUSTRY COOPERATION, Ulsan (KR); THE ASAN FOUNDATION, Seoul (KR)

(21) Appl. No.: 17/621,451

(22) PCT Filed: Jun. 22, 2020
(86) PCT No.: PCT/KR2020/008089

§ 371 Date: Dec. 21, 2021

(30) Foreign Application Priority Data
Jun. 21, 2019 (KR) 10-2019-0074073

(51) Int. Cl. G01N 33/68; C12Q 1/68

(52) U.S. Cl. CPC G01N 33/68; G01N 2333/435; G01N 2800/245; C12Q 1/68

(57) ABSTRACT

The present invention relates to a biomarker for the non-invasive diagnosis of kidney transplantation rejection and use thereof, and particularly to: a biomarker composition and kit for diagnosing antibody-mediated rejection after kidney transplantation or predicting the prognosis of a patient after kidney transplantation, comprising any one or more proteins selected from the group consisting of LBP and CST3, or gene(s) encoding for the same; a method for providing information required for diagnosing antibody-mediated rejection after kidney transplantation or predicting the prognosis of a patient after kidney transplantation, by using the marker composition; a method for providing information required for determining a therapy for rejection after kidney transplantation; a method for the diagnosis and treatment of antibody-mediated rejection after kidney transplantation; and a method for screening for a therapeutic agent for antibody-mediated rejection after kidney transplantation.

### TECHNICAL FIELD

The present invention relates to a biomarker associated with antibody-mediated rejection (ABMR) after kidney transplantation, a composition and a kit for diagnosing antibody-mediated rejection or predicting a prognosis for rejection after kidney transplantation, and a method for providing information for diagnosing antibody-mediated rejection or predicting a prognosis of rejection after kidney transplantation using the same.

### BACKGROUND ART

The field of organ transplantation is slowly becoming a part of precision medicine. When the current standard immunosuppressive therapy is used, acute rejection still occurs in 15 to 20% of patients who have received kidney transplantation, and until now, if there is an increase in serum creatinine concentration, new proteinuria expression or the like, it is diagnosed through tissue biopsy. Since the renal condition at the time when there is an increase in serum creatinine concentration or new proteinuria expression is a condition in which inflammation is already quite advanced, there is a need for biomarkers that are capable of the early detection of subclinical acute rejection. It is known that the innate/acquired immune responses occur at the molecular level before they occur at the histological level. Therefore, the development of non-invasive biomarkers for pre-diagnosing and monitoring clinical conditions after kidney transplantation is an essential step for precision medicine.

In the past, rejection after kidney transplantation was classified into hyperacute, acute and chronic rejections according to the time of the occurrence of rejection, but recently, the Banff classification is mainly used, which classifies rejections according to the mechanism of development and the findings of a renal biopsy. Prior to the Banff classification, the classification of pathologic diagnosis of transplanted kidneys was very simple, and in particular, acute and chronic rejections were mainly classified according to the location of the infiltration of inflammatory cells, and when the rejection was observed in the interstitium, it was classified as cellular, and when it was infiltrated into blood vessels, it was classified as vascular. That is, from the current point of view, all except hyperacute rejection were diagnostic classifications for T cell-mediated rejection, and at the time, the existence of ABMR, which was not a hyperacute clinical case, was not recognized, and there was no diagnostic basis therefor. Currently, hyperacute rejection is understood as the most severe form of acute ABMR.

Regarding the diagnosis of rejection after kidney transplantation, urine biomarkers for diagnosing acute rejection after kidney transplantation are known (Suthanthiran et al., Urinary-Cell mRNA Profile and Acute Cellular Rejection in Kidney Allografts. The New England Journal of Medicine (2013) 369:1). However, there are no studies to discover biomarkers that can differentiate antibody-mediated rejection from T cell-mediated rejection, and furthermore, to discover biomarkers that can differentiate and diagnose BK virus nephropathy and transplantation rejection, which are complications after kidney transplantation, and to utilize the same in the selection of treatment direction and the judgment of treatment progress after kidney transplantation.

### DISCLOSURE

### Technical Problem

As a result of diligent efforts to discover non-invasive and accurate diagnostic markers for kidney transplantation rejection, the inventors of the present invention identified new biomarkers that can predict the presence or absence of kidney transplantation rejection and specify the type of rejections through a proteomics approach, thereby completing the present invention.

By discovering markers for diagnosing kidney transplantation rejection or predicting a prognosis in urine exosomes of a group without major abnormalities after kidney transplantation (NOMOA), an antibody-mediated rejection (ABMR) group, a T cell-mediated rejection (TCMR) group and a BK virus infectious nephropathy group, the present invention aims to contribute to determining the progress of treatment after kidney transplantation and selecting an appropriate therapy.

### Technical Solution

In an aspect, the present invention provides a biomarker composition for diagnosing antibody-mediated rejection (ABMR) after kidney transplantation or predicting the prognosis of a patient after kidney transplantation, including any one or more proteins selected from the group consisting of LBP and CST3, or a gene encoding the same.

In another aspect, the present invention provides a composition for diagnosing antibody-mediated rejection after kidney transplantation or predicting the prognosis of a patient after kidney transplantation, including a material for measuring the expression level of any one or more proteins selected from the group consisting of LBP and CST3, or mRNA of a gene encoding the protein.

According to a preferred exemplary embodiment of the present invention, the material for measuring the expression level of mRNA may be a primer or a probe that specifically binds to the gene.

According to another preferred exemplary embodiment of the present invention, the material for measuring the expression level of the protein may be an antibody, an interacting protein, a ligand, an oligopeptide, peptide nucleic acid (PNA), nanoparticles or an aptamer that specifically binds to the protein or a fragment thereof.

In still another aspect, the present invention provides a kit for diagnosing antibody-mediated rejection after kidney transplantation or predicting the prognosis of a patient after kidney transplantation, including the above-described composition.

In still another aspect, the present invention provides a method for providing information required for diagnosing antibody-mediated rejection after kidney transplantation or predicting the prognosis of a patient after kidney transplantation, including measuring the expression level of any one or more proteins selected from the group consisting of LBP and CST3, or mRNA of a gene encoding the same in a sample isolated from a subject.

According to a preferred exemplary embodiment of the present invention, the sample may be urine or exosomes derived from urine.

According to another preferred exemplary embodiment of the present invention, the method may further include determining as antibody-mediated rejection, if the result of comparing the measured protein or mRNA expression level with a sample of a normal control group that did not receive kidney transplantation or a sample of a patient group that did not show rejection or BK virus nephropathy after kidney transplantation corresponds to any one or more selected from the group consisting of a) and b) below:

a) upregulation of LBP; and b) upregulation of CST3.

In still another aspect, the present invention provides a method for providing information required for determining a therapy for rejection after kidney transplantation, including measuring the expression level of any one or more proteins selected from the group consisting of LBP and CST3, or mRNA of a gene encoding the same in a sample isolated from a subject.

According to a preferred exemplary embodiment of the present invention, the sample may be urine or exosomes derived from urine.

According to another preferred exemplary embodiment of the present invention, the method may further include determining as antibody-mediated rejection and determining to apply a therapy for antibody-mediated rejection, if the result of comparing the measured protein or mRNA expression level with a sample of a normal control group that did not receive kidney transplantation or a sample of a patient group that did not show rejection or BK virus nephropathy after kidney transplantation corresponds to any one or more selected from the group consisting of a) and b) below:

a) upregulation of LBP; and b) upregulation of CST3.

In still another aspect, the present invention provides a method for diagnosing and treating antibody-mediated rejection after kidney transplantation, including a) measuring the expression level of any one or more proteins selected from the group consisting of LBP and CST3, or mRNA of a gene encoding the same in a sample isolated from a subject; b) comparing the expression level measured in step a) with a sample of a normal control group that did not receive kidney transplantation or a sample of a patient group that did not show rejection or BK virus nephropathy after kidney transplantation; c) determining as antibody-mediated rejection after kidney transplantation, if, as a result of the comparison of step b), the expression level in the sample isolated from the subject of step a) is higher than the expression level of the sample of a normal control group that did not receive kidney transplantation or the sample of a patient group that did not show rejection or BK virus nephropathy after kidney transplantation; and d) applying a therapy for antibody-mediated rejection to the subject of step a).

According to a preferred exemplary embodiment of the present invention, the sample may be urine or exosomes derived from urine.

According to another preferred exemplary embodiment of the present invention, the therapy for antibody-mediated rejection of step d) may be any one or more selected from the group consisting of steroid, plasmapheresis (PP), intravenous immunoglobulin (WIG), an anti-CD20 antibody (rituximab), a lymphocyte-depleting antibody, a proteasome inhibitor, a Cl-inhibitor and a monoclonal antibody against complement factor 5.

In still another aspect, the present invention provides a method for screening a therapeutic agent for antibody-mediated rejection after kidney transplantation, including a) treating a candidate substance of a therapeutic agent for antibody-mediated rejection after kidney transplantation to a sample isolated from a subject exhibiting antibody-mediated rejection after kidney transplantation; and b) measuring the expression level of any one or more proteins selected from the group consisting of LBP and CST3, or mRNA of a gene encoding the same.

According to a preferred exemplary embodiment of the present invention, the sample may be urine or exosomes derived from urine.

According to another preferred exemplary embodiment of the present invention, the screening method may further include determining the candidate substance of step a) as a therapeutic agent for antibody-mediated rejection after kidney transplantation, if the expression level of any one or more proteins selected from the group consisting of LBP and CST3, or mRNA of a gene encoding the same, which was measured in step b), is downregulated compared to before treating the candidate substance.

### Advantageous Effects

Since the biomarkers provided in the present invention are specifically and remarkably increased in expression in patients exhibiting antibody-mediated rejection after kidney transplantation, it is possible to accurately and quickly diagnose whether kidney transplantation is rejected and the type of rejection through a composition, a kit or a detection method including a material for measuring the expression level of the biomarker protein of the present invention or a gene encoding the same.

In addition, since the biomarkers of the present invention are specifically expressed in patients with antibody-mediated rejection, differentially from patients with other types of transplantation rejection or BK virus nephropathy, which is common after transplantation, it is possible to accurately and quickly identify antibody-mediated rejection, and it is possible to provide information required to determine a treatment policy thereafter.

### DESCRIPTION OF DRAWINGS

FIG. 1 is a diagram showing the selection process of biomarkers representing the TCMR, ABMR and BKVN groups.

FIG. 2 is a diagram showing the results of confirming the proteins of the ABMR group in which the expression levels differ by more than 2 times when compared with the NOMOA group using a volcano plot.

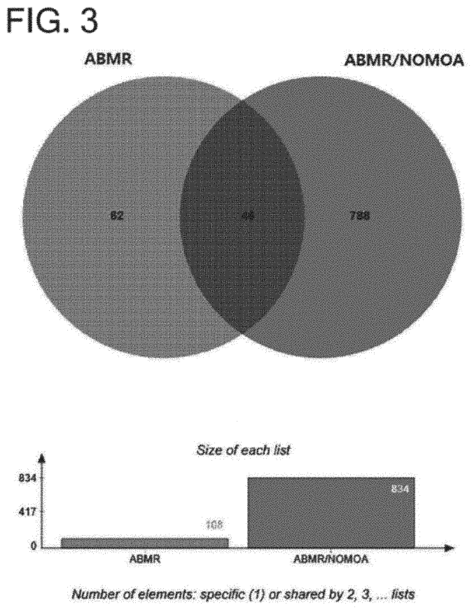

FIG. 3 is a diagram showing the results of selecting a total of 46 proteins present at the intersection site of a t-test result and a volcano plot result as biomarker candidate substances for ABMR.

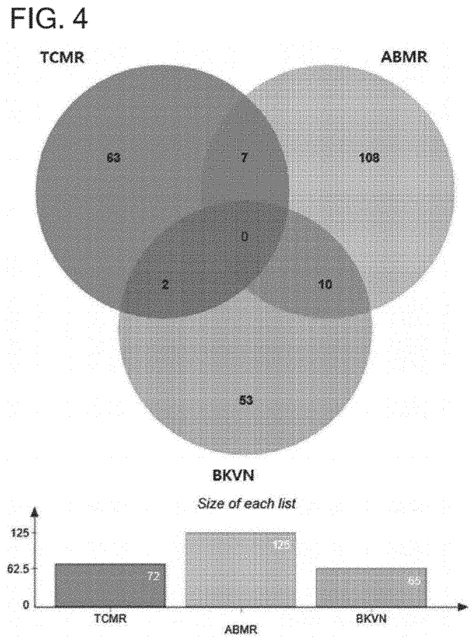

FIG. 4 shows the results of confirming that compared to the group without major abnormalities (NOMOA) after kidney transplantation and the donor group (DONOR), 63, 108 and 53 urine exosome proteins were significantly expressed differently from other groups in the T-cell mediated rejection group (TCMR), the antibody-mediated rejection group (ABMR) and the BK virus-infected nephropathy group (BKVN), respectively, by using a t-test.

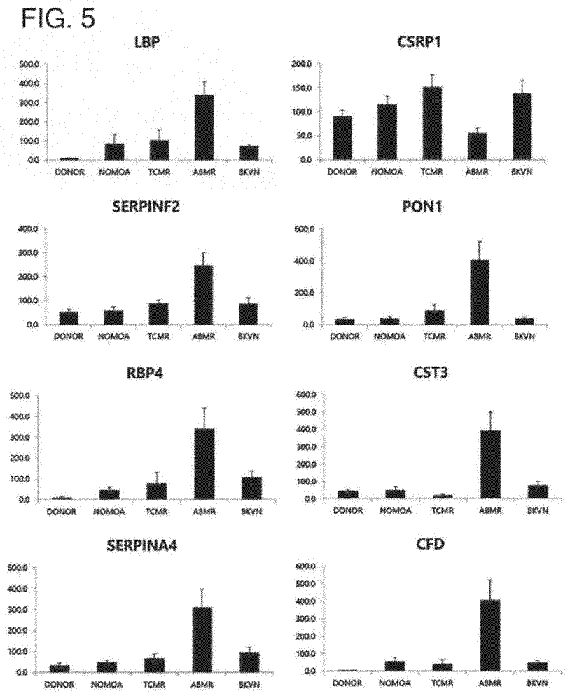

FIG. 5 is a diagram showing the results of the final selection of 8 proteins whose expression patterns were significantly different from those of the TCMR group and the BKVN group among the ABMR biomarker candidate substances.

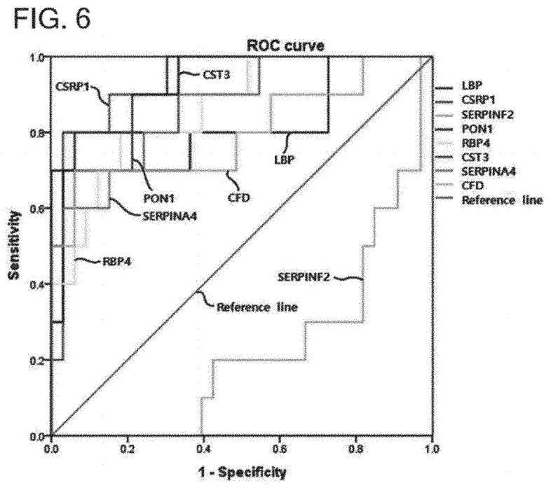

FIG. 6 is a result of performing ROC curve analyses by analyzing the expression levels of the 8 types of antibody-mediated rejection biomarkers selected in Example 4-4 of the present invention.

FIGS. 7a and 7b show the results of performing verification by western blot analyses for 2 types selected through an additional step among the 8 types of antibody-mediated rejection biomarkers selected in Example 4-4 of the present invention (7a: LBP and 7b: CST3 (cystatin C)).

FIG. 8 is a result of performing ROC curve analyses for the two biomarkers LBP and CST3 which were finally selected in Example 4-5 of the present invention.

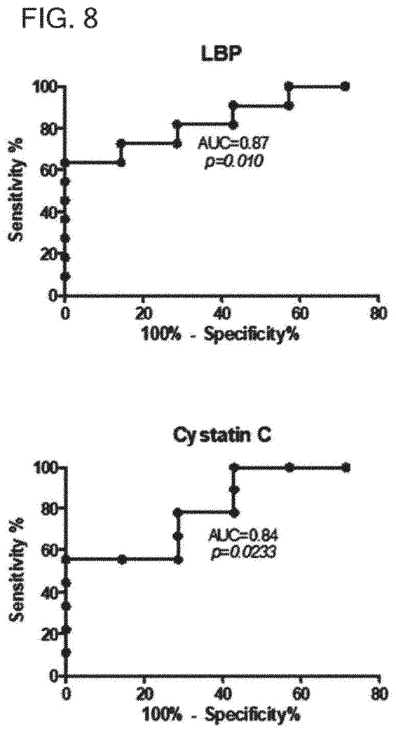

### BEST MODE

Hereinafter, the present invention will be described in more detail.

In an exemplary embodiment of the present invention, candidate proteins were selected based on proteomic analysis for the discovery of specific exosome proteins in urine samples of kidney transplantation patients and donors. In the quantitative analysis of urine exosome proteins, a total of 1,820 proteins were detected, of which 46 proteins having markedly different expression profiles between the “NOMOA and DONOR groups” and the “ABMR group” were selected. Finally, 8 proteins showing significantly different expression patterns from the TCMR group or the BKVN group were identified among the 46 proteins. As shown in the following [Table 2], the identified proteins were lipopolysaccharide-binding protein (LBP, UniProt Accession No. P18428), cysteine and glycine-rich protein 1 (CSRP1, UniProt Accession No. P21291), alpha-2-antiplasmin (SERPINF2, UniProt Accession No. P08697), retinol-binding protein 4 (RBP4, UniProt Accession No. P02753), cystatin-C(CST3, UniProt Accession No. P01034), complement factor D (CFD, UniProt Accession No. P00746), kallistatin (SERPINA4, UniProt Accession No. P29622) and serum paraoxonase/arylesterase 1 (PON1, UniProt Accession No. P27169).

In an additional exemplary embodiment of the present invention, verification was performed by western blot analysis on lipopolysaccharide-binding protein (LBP) and cystatin C (CST3) selected through an additional step among the 8 biomarkers. As a result, it was confirmed that LBP and CST3 were expressed significantly higher in the antibody-mediated rejection group (ABMR) compared to the normal control group that did not receive kidney transplantation and/or the group without major abnormalities after kidney transplantation (NOMOA), and the statistical significance thereof was also confirmed.

As such, the present invention provides a biomarker composition for diagnosing antibody-mediated rejection (ABMR) after kidney transplantation or predicting the prognosis of a patient after kidney transplantation, including the selected biomarkers involved in poor prognosis after kidney transplantation, that is, any one or more proteins selected from the group consisting of LBP and CST3, or a gene encoding the same. By using the same, since it is possible to predict the prognosis of a patient after kidney transplantation and to determine an appropriate treatment direction according to the predicted prognosis, it is possible to provide a customized therapy for the patient and to reduce the mortality rate of kidney transplantation patients with poor prognosis.

Renal allograft rejection refers to a series of immune responses that occur when the recipient's immune system to which the donor's kidney is transplanted recognizes the transplanted kidney as an external antigen, and it may be classified into antibody-mediated rejection (ABMR), T cell-mediated rejection (TCMR) and mixed rejection in which all of these appear in combination, according to the mechanism of development and the findings of a renal biopsy.

Since the biomarkers discovered in the present invention are proteins that are specifically highly expressed in antibody-mediated rejection, these are useful for diagnosing antibody-mediated rejection, including acute antibody-mediated rejection (CAMR) or chronic active antibody-mediated rejection.

As used herein, the term “biomarker” refers to a molecule that is quantitatively or qualitatively associated with the presence of a biological phenomenon, and the biomarker of the present invention refers to a protein or a gene encoding the same, which are capable of confirming whether there is antibody-mediated rejection after kidney transplantation, or it refers to a protein or a gene encoding the same that is a criterion for predicting a patient with good or poor prognosis. Biomarkers may be derived from genomic nucleotide sequences or from expressed nucleotide sequences (e.g., from RNA, nRNA, mRNA, cDNA, etc.) or from encoded polypeptides. This term includes nucleic acid sequences that are complementary to or flanked by a marker sequence, such as nucleic acids used as probes or primer pairs capable of amplifying the marker sequence.

According to an exemplary embodiment of the present invention, by confirming that the biomarkers were specifically highly expressed in the urine of patients showing antibody-mediated rejection (ABMR) compared to the group with no major abnormality (NOMOA) among kidney transplantation patients, it was found that the biomarkers may be used as diagnostic markers for antibody-mediated rejection.

As used herein, the term ‘expression’ means that a protein or nucleic acid is produced in a cell. The term ‘protein’ is used interchangeably with ‘polypeptide’ or ‘peptide’, and for example, it refers to a polymer of amino acid residues as commonly found as proteins in a natural state. ‘Polynucleotide’ or ‘nucleic acid’ refers to deoxyribonucleotide (DNA) or ribonucleotide (RNA) in a single- or double-stranded form. Unless otherwise limited, it also includes known analogs of natural nucleotides that are hybridized to nucleic acids in a manner similar to those of naturally occurring nucleotides. The term ‘mRNA’ refers to RNA that transfers genetic information (gene-specific nucleotide sequence) to ribosomes that specify amino acid sequences from specific genes during protein synthesis.

As used herein, the term ‘diagnosis’ means confirming the presence or characteristics of a pathological condition. The diagnosis in the present invention is to determine the presence or occurrence of a pathology of antibody-mediated rejection after kidney transplantation by measuring the level of any one or more proteins selected from the group consisting of LBP and CST3 or mRNA.

As used herein, the term “predicting a prognosis” means preliminarily considering and estimating the medical outcome, and for the purposes of the present invention, it means to preliminarily consider the course of the disease of a patient receiving kidney transplantation (pathology, improvement, transplantation rejection and drug resistance). In addition, the prognosis includes a positive prognosis (positive prognosis) or a negative prognosis (negative or poor prognosis), and in the present invention, a positive prognosis indicates that rejection or related symptoms or diseases did not occur after kidney transplantation, and a negative prognosis indicates that rejection or related symptoms occurred after kidney transplantation, and the type of rejection was antibody-mediated rejection.

The present invention also provides a composition for diagnosing antibody-mediated rejection after kidney transplantation or predicting the prognosis of a patient after kidney transplantation, including a material for measuring the expression level of mRNA of the two biomarkers of the present invention or the protein thereof.

When the composition for diagnosing or predicting a prognosis of the present invention is for measuring the expression level of mRNA, the material for measuring the mRNA expression level may be a probe or a primer set that specifically binds to a gene encoding any one or more proteins selected from the group consisting of LBP and CST3 or mRNA thereof.

As used herein, the term “primer” refers to a short nucleic acid sequence which may form a complementary template and base pairs as a nucleic acid sequence having a short free 3′ hydroxyl group, and serves as a starting point for copying the template. In the present invention, PCR amplification may be performed using the sense and antisense primers of the genes encoding the two biomarker proteins described above to diagnose the rejection of kidney transplantation or predict the prognosis through determining whether the desired product is generated. The PCR conditions and the length of the sense and antisense primers may be modified based on those known in the art.

As used herein, the term “probe” refers to a nucleic acid fragment such as RNA, DNA or the like corresponding to a short number of several bases to a long number of several hundred bases, which are capable of specifically binding to mRNA, and since it is labeled, it is possible to confirm the presence or absence of specific mRNA. The probe may be constructed in the form of an oligonucleotide probe, a single-stranded DNA probe, a double-stranded DNA probe, an RNA probe or the like. In the present invention, hybridization may be performed using a probe complementary to the mRNA of the gene encoding the biomarker protein of the present invention to diagnose antibody-mediated rejection reaction after kidney transplantation or to predict the prognosis through hybridization. Selection of suitable probes and conditions for hybridization may be modified based on those known in the art.

The primer or probe of the present invention may be chemically synthesized using the phosphoramidite solid-support method or other well-known method. Such nucleic acid sequences may also be modified using a number of means known in the art. Non-limiting examples of such modifications include methylation, encapsulation, substitution of one or more homologs of natural nucleotides and modifications between nucleotides, for example, to uncharged linkers (e.g., methyl phosphonate, phosphotriester, phosphoroamidates, carbamates, etc.) or charged linkers (e.g., phosphorothioates, phosphorodithioates, etc.).

Meanwhile, in the present invention, the material for measuring the expression level of the biomarker protein may be an antibody that specifically binds to any one or more proteins selected from the group consisting of LBP and CST3.

The term “antibody” is a term known in the art and refers to a specific protein molecule directed against an antigenic site. For the purposes of the present invention, an antibody refers to an antibody that specifically binds to the biomarker protein of the present invention, and for such an antibody, by cloning each gene into an expression vector according to a conventional method to obtain a protein encoded by the biomarker gene, it may be prepared from the obtained protein by a conventional method. This includes partial peptides that may be made from the protein, and the partial peptides of the present invention include at least 7 amino acids, preferably, 9 amino acids, and more preferably, 12 or more amino acids. The form of the antibody of the present invention is not particularly limited, and a polyclonal antibody, a monoclonal antibody or a part thereof is also included in the antibody of the present invention as long as it has antigen-binding property, and all immunoglobulin antibodies are included. Furthermore, the antibody of the present invention also includes special antibodies such as humanized antibodies and the like. The antibody against the protein encoded by the biomarker gene of the present invention may be any antibody that may be prepared by a method known in the art. For example, the antibody used for the diagnosis of antibody-mediated rejection after kidney transplantation of the present invention or the detection of a predictive prognostic marker may include a complete form having two full-length light chains and two full-length heavy chains, as well as a functional fragment of the antibody molecule. The functional fragment of the antibody molecule refers to a fragment having at least an antigen-binding function and may be Fab, F(ab′), F(ab′)2, Fv or the like, but is not particularly limited thereto.

In another aspect, the present invention provides a kit for diagnosing antibody-mediated rejection after kidney transplantation or predicting the prognosis of a patient after kidney transplantation, including the above-described composition for diagnosing antibody-mediated rejection after kidney transplantation or predicting the prognosis of a patients after kidney transplantation.

According to a preferred exemplary embodiment of the present invention, the kit may be an RT-PCR kit, a competitive RT-PCR kit, a real-time RT-PCR kit, a DNA chip kit or a protein chip kit.

The kit of the present invention may include an antibody recognising the two types of biomarker proteins or a primer and a probe recognizing mRNA of genes encoding the biomarker proteins, as well as a composition, a solution or a device including one or more other components suitable for an analysis method.

In a specific aspect, the kit may be a diagnostic kit, which is characterized in that it includes essential elements necessary to perform a reverse transcription polymerase reaction. The reverse transcription polymerase reaction kit includes each primer pair specific for the gene encoding the biomarker protein. The primer is a nucleotide having a sequence specific to the nucleic acid sequence of each gene, and is about 7 bp to 50 bp in length, and more preferably, about 10 bp to 30 bp in length. In addition, a primer specific to the nucleic acid sequence of the control group gene may be included. Other reverse transcription polymerase reaction kits may include test tubes or other suitable containers, reaction buffers (at various pH and magnesium concentrations), deoxynucleotides (dNTPs), enzymes such as Taq-polymerase and reverse transcriptase, DNAse, RNAse inhibitor, DEPC-water, sterilized water and the like.

In still another aspect, it may be a diagnostic kit, which is characterized in that it includes essential elements necessary to perform the DNA chip. The DNA chip kit may include a substrate to which cDNA or oligonucleotide corresponding to a gene or fragment thereof is attached, and reagents, agents, enzymes and the like for constructing a fluorescently labeled probe. In addition, the substrate may include cDNA or oligonucleotide corresponding to the control group gene or a fragment thereof.

In still another aspect, the kit for measuring the expression level of a protein in the present invention may include a substrate, an appropriate buffer solution, a secondary antibody labeled with a color development enzyme or a fluorescent material, a color development substrate and the like for immunological detection of the antibody. For the substrate in the above, a nitrocellulose membrane, a 96-well plate synthesized from a polyvinyl resin, a 96-well plate synthesized from a polystyrene resin, a glass slide glass and the like may be used, and peroxidase and alkaline phosphatase may be used as the color development enzyme. In addition, FTTC, RITC or the like may be used as the fluorescent material, and 2,2′-azino-bis-(3-ethylbenzothiazoline-6-sulfonic acid) (ABTS), o-phenylenediamine (OPD), tetramethyl benzidine (TMB) or the like may be used as the color development substrate solution.

In still another aspect, the present invention provides a method for providing information required for diagnosing antibody-mediated rejection after kidney transplantation or predicting the prognosis of a patient after kidney transplantation, including measuring the expression level of any one or more proteins selected from the group consisting of LBP and CST3, or mRNA of a gene encoding the same in a sample isolated from a subject.

As used herein, the term “subject” is an individual who has received kidney transplantation, and it may include mammals such as humans, primates including chimpanzees, pets such as dogs and cats, domestic animals such as cattle, horses, sheep and goats, and rodents such as mice and rats, formed fish and the like that may or have had rejection after kidney transplantation without limitation.

As used herein, the term “sample” used for analysis includes a biological sample capable of identifying proteins specific for antibody-mediated rejection after kidney transplantation that can be distinguished from normal conditions, such as blood, plasma, serum, saliva, nasal fluid, sputum, ascites, vaginal secretion, urine, feces and the like. Preferably, it may be a biological liquid sample, for example, blood, serum, plasma or urine, and most preferably, urine or exosomes derived from urine. The sample may be prepared to increase the detection sensitivity of the protein marker, and for example, the sample obtained from the patient may be pretreated using a method such as anion exchange chromatography, affinity chromatography, size exclusion chromatography, liquid chromatography, sequential extraction, gel electrophoresis or the like.

As used herein, the “measurement of the expression level of a protein” is a process of confirming the presence and expression level of biomarker proteins expressed in a biological sample in order to diagnose antibody-mediated rejection after kidney transplantation or predict the prognosis of a patient who has received kidney transplantation, and it is possible to detect the presence of the protein or measure the amount of the protein using an antibody that specifically binds to the protein. Antibodies specific to the protein are as described in the composition for diagnosing or predicting the prognosis of the present invention. For the method of measuring the expression level of a protein, methods known in the art may be used without limitation, for example, western blotting, dot blotting, enzyme-linked immunosorbent assay (ELISA), radioimmunoassay (RIA), radioimmunodiffusion, Ouchterlony immtmodiffusion, rocket immunoelectrophoresis, immunohistochemical staining, immunoprecipitation, complement fixation assay, flow cytometry (FACS), protein chip method or the like, but is not limited thereto.

As used herein, the “measurement of the expression level of mRNA” is a process of confirming the presence and expression level of mRNA of a gene encoding biomarker proteins in a biological sample for diagnosing antibody-mediated rejection after kidney transplantation or predicting the prognosis of a patient who has received kidney transplantation, and it may be determined by measuring the amount of mRNA. Analysis methods therefor include RT-PCR, competitive RT-PCR, real-time RT-PCR, RNase protection method, northern blotting, DNA chip technology or the like, but is not limited thereto.

The method for providing information required for diagnosing antibody-mediated rejection after kidney transplantation or predicting the prognosis of a patient after kidney transplantation according to the present invention may further include determining as antibody-mediated rejection, if the result of comparing the measured protein or mRNA expression level with a sample of a normal control group that did not receive kidney transplantation or a sample of a patient group that did not show rejection or BK virus nephropathy after kidney transplantation corresponds to any one or more selected from the group consisting of a) and b) below:

a) upregulation of LBP; and

b) upregulation of CST3.

The normal control group means a normal person who has not received kidney transplantation and may be interpreted as including a kidney donor (donor). The patient group which did not show rejection or BK virus infection nephropathy after kidney transplantation refers to a kidney transplantation patient who received a kidney transplant, but showed stable kidney function and did not exhibit antibody-mediated rejection, T-cell-mediated rejection, mixed rejection or BK virus infection nephropathy. By obtaining and comparing the expression levels or expression patterns of proteins or genes encoding the same in samples collected from these normal control group and patients and samples collected from patients who want to know the presence or prognosis of the rejection of kidney transplantation, it is possible to accurately predict the prognosis of a patient who wants to know the prognosis.

Furthermore, the method for providing information required for diagnosing antibody-mediated rejection after kidney transplantation or predicting the prognosis of a patient after kidney transplantation according to the present invention is capable of specifically diagnosing antibody-mediated rejection by differentiating from T cell-mediated rejection and BK virus nephropathy, and thus, by obtaining and comparing the expression levels or expression patterns of proteins or genes encoding the same in samples collected from a patient group showing T cell-mediated rejection and/or BK virus nephropathy and samples collected from patients who want to know the presence or prognosis of the rejection of kidney transplantation, it is possible to clearly identify the cause and type of rejection.

Therefore, according to the present invention, it is possible to accurately diagnose or predict the type and prognosis of rejection after kidney transplantation, and it is possible to achieve an advantage of being able to establish an appropriate treatment plan according to the diagnosis or predicted prognosis.

Accordingly, the present invention provides a method for providing information required for determining a therapy for rejection after kidney transplantation, including measuring the expression level of any one or more proteins selected from the group consisting of LBP and CST3, or mRNA of a gene encoding the same in a sample isolated from a subject.

The method for providing information required for determining a therapy for rejection after kidney transplantation according to the present invention may further include determining as antibody-mediated rejection and determining to apply a therapy for antibody-mediated rejection, if the result of comparing the measured protein or mRNA expression level with a sample of a normal control group that did not receive kidney transplantation or a sample of a patient group that did not show rejection or BK virus nephropathy after kidney transplantation corresponds to any one or more selected from the group consisting of a) and b) below:

a) upregulation of LBP; and

b) upregulation of CST3.

In the method for providing information required for determining a therapy for rejection after kidney transplantation according to the present invention, after it is determined to apply a therapy for antibody-mediated rejection, various known therapies for antibody-mediated rejection may be appropriately applied alone or in combination to the patient.

For example, steroid, plasmapheresis (PP), intravenous immunoglobulin (IVIG), an anti-CD20 antibody, a lymphocyte-depleting antibody, a proteasome inhibitor (bortezomib), a Cl-inhibitor, a monoclonal antibody against complement factor 5 (eculizumab) and the like, which are recommended by the guidelines of the Kidney Disease Improving Global Outcomes (KDIGO), may be used alone or in combination. Most of the treatment policies are two major treatment strategies: 1) plasmapheresis to remove antibodies, and treatment to modulate B cells and immune function, but are not limited thereto, and an appropriate therapy may be selected under the judgment of a person skilled in the art or a clinician, and therapies such as bortezomib, eculizumab or the like may be additionally considered.

Accordingly, in an additional aspect, the present invention provides a method for diagnosing and treating antibody-mediated rejection after kidney transplantation.

Specifically, the method for diagnosing and treating antibody-mediated rejection after kidney transplantation according to the present invention includes the following steps:

a) measuring the expression level of any one or more proteins selected from the group consisting of LBP and CST3, or mRNA of a gene encoding the same in a sample isolated from a subject;

b) comparing the expression level measured in step a) with a sample of a normal control group that did not receive kidney transplantation or a sample of a patient group that did not show rejection or BK virus nephropathy after kidney transplantation;

c) determining as antibody-mediated rejection after kidney transplantation, if, as a result of the comparison of step b), the expression level in the sample isolated from the subject of step a) is higher than the expression level of the sample of a normal control group that did not receive kidney transplantation or the sample of a patient group that did not show rejection or BK virus nephropathy after kidney transplantation; and

d) applying a therapy for antibody-mediated rejection to the subject of step a).

In the method for diagnosing and treating antibody-mediated rejection after kidney transplantation, the therapy for antibody-mediated rejection in step d) may be any one or more selected from the group consisting of steroid, plasmapheresis (PP), intravenous immunoglobulin (WIG), an anti-CD20 antibody and a lymphocyte-depleting antibody, but is not limited thereto, and as described above, an appropriate therapy may be selected under the judgment of a person skilled in the art or a clinician.

In another aspect, the present invention provides a method for screening a therapeutic agent for antibody-mediated rejection after kidney transplantation, including a) treating a candidate substance of a therapeutic agent for antibody-mediated rejection after kidney transplantation to a sample isolated from a subject exhibiting antibody-mediated rejection after kidney transplantation; and b) measuring the expression level of any one or more proteins selected from the group consisting of LBP and CST3, or mRNA of a gene encoding the same.

The method for screening a therapeutic agent for antibody-mediated rejection after kidney transplantation according to the present invention may further include c) determining the candidate substance of step a) as a therapeutic agent for antibody-mediated rejection after kidney transplantation, if the expression level of any one or more proteins selected from the group consisting of LBP and CST3, or mRNA of a gene encoding the same, which was measured in step b), is downregulated compared to before treating the candidate substance.

Specifically, it is a method of comparing an increase or decrease in the mRNA or protein expressions of markers in the presence and absence of candidate substances for treating antibody-mediated rejection after kidney transplantation, which may be effectively used for screening therapeutic agents for antibody-mediated rejection after kidney transplantation.

That is, the expression levels of the two biomarker proteins of the present invention or genes encoding the same are measured in biological samples isolated from patients who exhibited antibody-mediated rejection after kidney transplantation in the absence of the candidate substances for treating antibody-mediated rejection after kidney transplantation, and in addition, the expression levels of the two biomarker proteins of the present invention or genes encoding the same are measured in the presence of the therapeutic candidate substances, and after comparing both cases, it is possible to select the substances that reduce the expression levels in the presence of the therapeutic candidate substances compared to the expression levels in the absence thereof as therapeutic agents for antibody-mediated rejection after kidney transplantation.

The method for providing information required for diagnosing antibody-mediated rejection after kidney transplantation or predicting the prognosis of a patient after kidney transplantation, the method for diagnosing and treating antibody-mediated rejection after kidney transplantation and the method for screening a therapeutic agent for antibody-mediated rejection after kidney transplantation according to the present invention may all include an in vitro method performed on samples isolated from subjects.

However, when the method for screening a therapeutic agent for antibody-mediated rejection is performed in vivo, it is intended for mammals excluding humans, and it is not intended for humans or the human body.

Since other specific contents for the method for providing information required for determining a therapy for rejection after kidney transplantation, the method for diagnosing and treating antibody-mediated rejection after kidney transplantation and/or the method for screening a therapeutic agent for antibody-mediated rejection after kidney transplantation according to the present invention are the same as the method for providing information required for diagnosing antibody-mediated rejection after kidney transplantation or predicting the prognosis of a patient after kidney transplantation, the descriptions thereof will be omitted.

### MODES OF THE INVENTION

Hereinafter, the present invention will be described in more detail through Examples. These Examples are for illustrative purposes only, and it will be apparent to those of ordinary skill in the art that the scope of the present invention is not interpreted to be limited by these Examples.

### Example 1

Selection of Patients and Preparation of Urine Samples

In order to discover biomarkers for diagnosing antibody-mediated rejection in kidney transplantation patients, mine samples were collected 2 to 3 hours before the biopsy from a total of 60 patients, including patients and donors who underwent an indication biopsy within 5 years of kidney transplantation. In addition, urine samples were collected from living kidney donors immediately before kidney donation surgery. The patients were classified into 5 groups according to the pathological diagnosis of biopsy: 12 antibody-mediated rejection (ABMR) groups, 8 T cell-mediated rejection (ABMR) groups, 5 BK virus nephropathy (BKVN) groups, 11 groups without major abnormalities (NOMOA) and 24 donor groups (DONOR). Urine from patients with mixed allopathic lesions was excluded. This study was conducted with the approval of the Medical Institution Evaluation Committee at Asan Medical Center, and written consent was obtained from all patients.

### Example 2

Processing of Urine Samples and Separation of Exosomes

By slightly modifying the methods of Pisitkim et al. (2004) and Alvarez et al. (2012), exosomes were separated from the prepared urine samples by step-wise ultracentrifugation. Specifically, 30 mL or more of urine samples was collected from each experiment participant, and 400 μL of a protease inhibitor mixture [50 μM 4-(2-animoethyl) benzenesulfonyl fluoride hydrochloride (AEBSF-HCl, Sigma Aldrich), 2 μM leupeptin-hemisulfate (Sigma Aldrich) and 3.3 mM sodium azide (Sigma Aldrich)] was added to each urine sample. In order to remove urinary sediments including whole cells, large membrane particles and other debris, the urine samples were centrifuged at 4,000 rpm and 4° C. for 15 minutes, and stored at −80° C. until the exosomes were extracted and used in the experiment.

Afterwards, the frozen urine samples were thawed by 15 mL each, vortexed for 1 minute and then centrifuged at 17,000×g for 15 minutes at room temperature, and the supernatant (SN1) was collected. SN1 was ultracentrifuged at 200,000×g for 70 min at room temperature using a Beckman Coulter Optima L-80xp ultracentrifuge, rotor SW40Ti (Beckman Coulter, Brea, Calif., USA). The supernatant was discarded, and the pellet was washed by dissolving in 11 mL of DPBS, followed by ultracentrifugation again at 200,000×g for 70 minutes at room temperature. The supernatant was discarded and the exosome pellet was used for protein separation or exosome particle quantification.

### Example 3

LC-MS Analysis and Protein Identification

Sample preparation for proteomic analysis was subjected to freeze drying, protein solubilization and digestion. The resulting peptide mixture was desalted, dried using C18 reverse phase chromatography, and then analyzed by high resolution mass spectrometry combined with nano-flow liquid chromatography.

Sequence database analysis and label free quantitation (LFQ) analysis were applied using Proteome Discoverer 2.2 (Thermo Fisher Scientific) to search for biomarker candidate substances for diagnosing or predicting kidney transplantation rejection. From this, 1,820 urine exosome proteins were identified. Gene ontology assignments, molecular function and Kegg pathway analysis were performed using the DAVID bioinformation database.

### Example 4

Selection of Biomarker Candidates

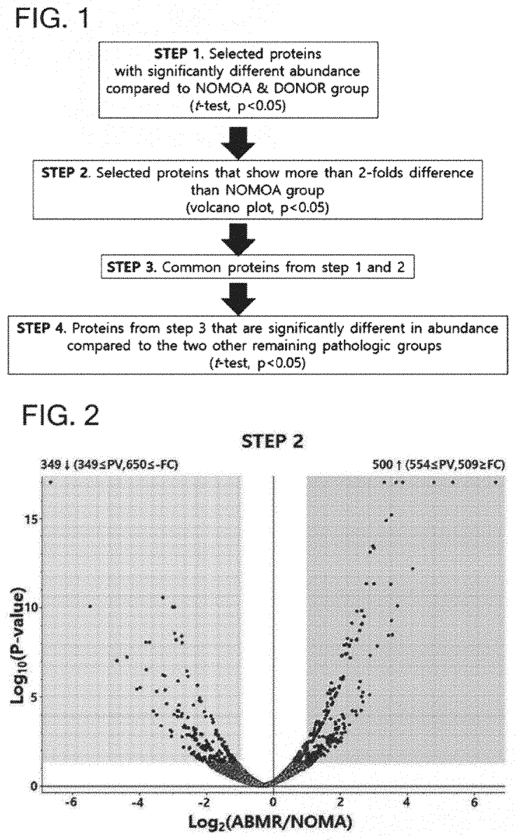

The process of selecting proteins as biomarker candidate substances is summarized in [FIG. 1]. In order to select specific biomarkers representing each pathological group, a t-test was used to select proteins with significantly different average abundances compared to the NOMOA and DONOR groups. In addition, a volcano plot was used to select proteins showing a two-fold or more difference compared to the NOMOA group. The overlapping proteins were selected from the two analyses and compared again with the two other pathological groups to compare the significant difference in average abundances. All analyses were performed with a cut off of p<0.05.

4-1. Identification of Protein Biomarkers and Primary Screening of ABMR Patients

It was attempted to select specific biomarkers representing each pathological group from 4,193 urine proteins, and sequence database analysis and label free quantitation (LFQ) analysis were applied using Proteome Discoverer 2.2 (Thermo Fisher Scientific) to identify 1,820 urine exosome proteins.

In order to select markers which are capable of distinguishing the ABMR group among the 1,820 urine exosome proteins, first, as a result of primary selection of proteins with significantly different average abundances in the ABMR group compared to the NOMOA and Donor groups using the T test, it was confirmed that 108 proteins were specifically expressed only in the ABMR group.

Next, as a result of identifying proteins showing a two-fold or more difference in the expression levels by comparing only the NOMOA group and the ABMR group, 834 proteins were selected.

Among the 108 proteins which were primarily selected and the 834 proteins showing a two-fold or more difference in the expression levels, 46 overlapping proteins were finally selected as ABMR marker candidates, and the results of comparing the significant difference in the abundance ratios of the TCMR group and the BKVN group again are shown in [Table 1] below.

TABLE 1
List of proteins showing differences between NOMOA, DONOR and ABMR groups (46 types)

| | | | Abundance | | | | | |
|---|---|---|---|---|---|---|---|---|
| | | Abundance | Ratio | T test | T test | T test | T test | |
| | Protein FDR | Ratio: | P-Value: | NOMOA | ABMR | ABMR | ABMR | |
| Registration | Confidence: | (ABMR)/ | (ABMR)/ | vs | vs | vs | vs | |
| No. | Combined | (NOMOA) | (NOMOA) | ABMR | TCMR | BKVN | donor | Description |
| Q07000 | High | 4.276 | 0.000125 | 0.001 | 0.267 | 0.525 | 0.051 | HLA class I histocompatibility antigen |
| O15144 | High | 0.423 | 0.018431 | 0.001 | 0.021 | 0.044 | 0.127 | Actin-related protein 2/3 complex subunit 2 |
| P01019 | High | 6.23 | 8.81E−10 | 0.003 | 0.007 | 0.324 | 0.002 | Angiotensinogen |
| Q9NZD2 | High | 0.273 | 0.022965 | 0.003 | 0.041 | 0.227 | 0.000 | Glycolipid transfer protein |
| P08697 | High | 3.009 | 0.000109 | 0.007 | 0.018 | 0.023 | 0.005 | Alpha-2-antiplasmin |
| P02652 | High | 4.085 | 9.45E−07 | 0.007 | 0.975 | 0.328 | 0.005 | Apolipoprotein A-II |
| P01008 | High | 2.679 | 0.00045 | 0.008 | 0.060 | 0.138 | 0.001 | Antithrombin-III |
| P62244 | High | 0.21 | 0.00477 | 0.008 | 0.129 | 0.122 | 0.003 | 40S ribosomal protein S15a |
| P02647 | High | 3.143 | 6.21E−05 | 0.009 | 0.019 | 0.651 | 0.001 | Apolipoprotein A-I |
| Q16563 | High | 0.378 | 0.009505 | 0.009 | 0.005 | 0.201 | 0.001 | Synaptophysin-like protein 1 |
| Q9UGM5 | High | 4.342 | 1.28E−08 | 0.010 | 0.580 | 0.053 | 0.085 | Fetuin-B |
| P18428 | High | 3.655 | 7.69E−06 | 0.011 | 0.020 | 0.004 | 0.001 | Lipopolysaccharide-binding protein |
| P21291 | High | 0.395 | 0.013813 | 0.012 | 0.013 | 0.045 | 0.047 | Cysteine and glycine-rich protein |
| P15291 | High | 3.045 | 9.35E−05 | 0.014 | 0.034 | 0.157 | 0.010 | Beta-1,4-galactosyltransferase 1 |
| P02753 | High | 3.902 | 2.92E−06 | 0.014 | 0.037 | 0.047 | 0.008 | Retinol-binding protein 4 |
| P54709 | High | 0.27 | 0.042444 | 0.016 | 0.154 | 0.272 | 0.030 | Sodium/potassium-transporting ATPase subunit |
| | | | | | | | | beta-3 |
| Q8WWT9 | High | 0.215 | 0.004434 | 0.016 | 0.004 | 0.250 | 0.001 | Solute carrier family 13 member 3 |
| P01034 | High | 4.863 | 1.24E−08 | 0.017 | 0.011 | 0.026 | 0.016 | Cystatin-C |
| P00746 | High | 6.285 | 7.42E−10 | 0.018 | 0.015 | 0.016 | 0.008 | Complement factor D |
| P29622 | High | 3.368 | 2.45E−05 | 0.018 | 0.026 | 0.048 | 0.013 | Kallistatin |
| P0DJI8 | High | 0.353 | 0.047116 | 0.020 | 0.498 | #DIV/0! | #DIV/0! | Serum amyloid A-1 protein |
| P21810 | High | 2.565 | 0.000742 | 0.020 | 0.255 | 0.968 | 0.101 | Biglycan |
| P14174 | High | 0.133 | 6.23E−09 | 0.022 | 0.085 | 0.124 | 0.048 | Macrophage migration inhibitory factor |
| P27169 | High | 4.935 | 6.45E−08 | 0.023 | 0.045 | 0.023 | 0.021 | Serum paraoxonase/arylesterase 1 |
| P00734 | High | 3.597 | 9.72E−06 | 0.023 | 0.078 | 0.041 | 0.013 | Prothrombin |
| Q15485 | High | 2.348 | 0.000814 | 0.027 | 0.405 | 0.388 | 0.046 | Ficolin-2 |
| Q06033 | High | 2.086 | 0.013977 | 0.031 | 0.015 | 0.053 | 0.036 | Inter-alpha-trypsin inhibitor heavy chain H3 |
| P05546 | High | 3.667 | 7.34E−06 | 0.033 | 0.123 | 0.067 | 0.019 | Heparin cofactor 2 |
| Q86Y46 | High | 0.253 | 0.00696 | 0.034 | 0.626 | #DIV/0! | 0.061 | Keratin, type II cytoskeletal 73 |
| Q92520 | High | 2.658 | 0.005751 | 0.034 | 0.232 | 0.630 | 0.027 | Protein FAM3C |
| P06681 | High | 3.37 | 2.43E−05 | 0.035 | 0.061 | 0.057 | 0.011 | Complement C2 |
| P04278 | High | 2.461 | 0.000239 | 0.037 | 0.442 | 0.061 | 0.025 | Sex hormone-binding globulin |
| Q9BZF9 | High | 0.472 | 0.049706 | 0.037 | 0.778 | 0.205 | 0.006 | Uveal autoantigen with coiled-coil domains and |
| | | | | | | | | ankyrin repeats |
| Q9Y512 | High | 0.402 | 0.043272 | 0.038 | 0.006 | 0.618 | 0.003 | Sorting and assembly machinery component 50 |
| | | | | | | | | homolog |
| Q92845 | High | 0.329 | 0.029713 | 0.038 | 0.011 | 0.423 | 0.000 | Kinesin-associated protein 3 |
| O14657 | High | 2.267 | 0.017737 | 0.039 | #DIV/0! | 0.056 | #DIV/0! | Torsin-1B |
| Q969L2 | High | 0.439 | 0.032975 | 0.040 | 0.024 | 0.108 | 0.000 | Protein MAL2 |
| P0DOY2 | High | 2.562 | 0.000752 | 0.041 | 0.191 | 0.183 | 0.003 | Immunoglobulin lambda constant 2 |
| P11678 | High | 0.242 | 0.023956 | 0.042 | 0.343 | #DIV/0! | 0.166 | Eosinophil peroxidase |
| P01860 | High | 4.256 | 7.56E−07 | 0.044 | 0.147 | 0.105 | 0.022 | Immunoglobulin heavy constant gamma 3 |
| Q99972 | High | 5.61 | 6.48E−09 | 0.046 | 0.053 | 0.144 | 0.038 | Myocilin |
| P20062 | High | 4.436 | 0.000954 | 0.046 | 0.912 | #DIV/0! | #DIV/0! | Transcobalamin-2 |
| P26572 | High | 3.104 | 6.07E−06 | 0.046 | 0.035 | 0.198 | 0.229 | Alpha-1,3-mannosyl-glycoprotein |
| | | | | | | | | 2-beta-N-acetylglucosaminyltransferase |
| O14896 | High | 4.395 | 0.000457 | 0.047 | 0.176 | 0.454 | 0.132 | Interferon regulatory factor 6 |
| P14543 | High | 2.464 | 0.001158 | 0.050 | 0.077 | 0.127 | 0.032 | Nidogen-1 |
| P00403 | High | 0.167 | 0.00137 | 0.050 | 0.473 | 0.331 | 0.185 | Cytochrome c oxidase subunit 2 |

4-2. Final Selection of ABMR-Specific Candidate Biomarkers

The final candidate proteins were selected based on the T test analysis in order to discover the exosome proteins specific to each of the ABMR, TCMR and BKVN groups. Among the urine exosome proteins, compared to the NOMOA group or the DONOR group, 46 proteins in [Table 1] were primarily selected among the proteins specifically expressed only in the ABMR group through the process of selecting only proteins having a two-fold or more difference in expression profiles, compared to the NOMOA group.

Among the 46 proteins which were primarily selected in [Table 1], proteins with significantly different average abundances only in the ABMR group compared to the TCMR group were selected as candidate biomarkers, and proteins with significantly different average abundances only in the ABMR group compared to the BKVN group were selected as candidate biomarkers, and finally, among the candidate biomarker proteins, 8 overlapping proteins were selected as ABMR biomarker proteins expected to show differences in the TCMR, BKVN and ABMR groups. As shown in the following [Table 2], the identified proteins were lipopolysaccharide-binding protein (LBP, UniProt Accession No. P18428), cysteine and glycine-rich protein 1 (CSRP1, UniProt Accession No. P21291), alpha-2-antiplasmin (SERPINF2, UniProt Accession No. P08697), retinol-binding protein 4 (RBP4, UniProt Accession No. P02753), cystatin-C(CST3, UniProt Accession No. P01034), complement factor D (CFD, UniProt Accession No. P00746), kallistatin (SERPINA4, UniProt Accession No. P29622) and serum paraoxonase/arylesterase 1 (PON1, UniProt Accession No. P27169).

4-3. Confirmation of the Characteristics of the Selected ABMR-Specific Biomarkers

In order to confirm the characteristics of the finally selected biomarkers, gene ontology assignments, molecular function and Kegg pathway analysis were performed using the DAVID bioinformation database, and the results are shown in [Table 2].

TABLE 2
Functional characteristics of selected ABMR-specific markers

| Registration | | | | | |
|---|---|---|---|---|---|
| No. | Description | Gene | BP direct | MF direct | Kegg pathway |
| P08697 | Alpha-2- | SERPINE2 | regulation of blood vessel | endopeptidase | Complement and |
| | antiplasmin | | size by renin-angiotensin | inhibitory activity | coagulation |
| | | | negative regulation of | protease binding | cascades |
| | | | fibrinolysis | serine-type | |
| | | | fibrinolysis | endopeptidase | |
| | | | positive regulation of | inhibitor activity | |
| | | | collagen biosynthetic | | |
| | | | process | | |
| | | | positive regulation of | | |
| | | | stress fiber assembly | | |
| | | | acute-phase response | | |
| | | | platelet degranulation | | |
| | | | negative regulation of | | |
| | | | endopeptidase activity | | |
| P18428 | Lipopoly- | LBP | opsonization | | |
| | saccharide- | | acute-phase response | | |
| | binding | | innate immune response | | |
| | protein | | | | |
| P21291 | Cystein and | CSRP1 | | | |
| | glycine- | | | | |
| | rich | | | | |
| | protein 1 | | | | |
| P02753 | Retinol- | RBP4 | eye development | | |
| | binding | | retinoid metabolic process | | |
| | protein 4 | | | | |
| P01034 | Cystatin-C | CST3 | negative regulation of | cystein-type | |
| | | | proteolysis | endopeptidase | |
| | | | eye development | inhibitor activity | |
| | | | response to nutrient levels | beta-amyloid | |
| | | | cellular protein metabolic | binding | |
| | | | process | protease binding | |
| | | | | endopeptidase | |
| | | | | inhibitor activity | |
| P00746 | Complement | CFD | complement activation | serine-type | Complement and |
| | factor D | | platelet degranulation | endopeptidase | coagulation |
| | | | proteolysis | activity | cascades |
| P29622 | Kallistatin | SEPINA4 | platelet degranulation | serine-type | |
| | | | negative regulation of | endopeptidase | |
| | | | endopeptidase activity | inhibitor activity | |
| P27169 | Serum | PON1 | response to nutrient levels | phospholipid | |
| | paraoxonase/ | | cholesterol metabolic | binding | |
| | arylesterase 1 | | process | | |

As shown in Table 2 above, it can be seen that the selected ABMR-specific markers were associated with complement activation, platelet degranulation and innate immune response, and among the selected markers, the functional characteristics of CSRP1 have not been reported yet.

4-4. Confirmation of Accuracy of ABMR-Specific Biomarkers

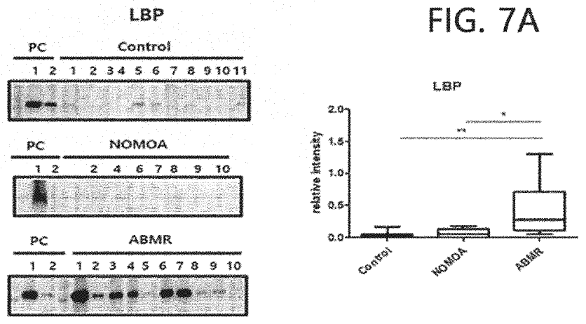

By performing ROC analyses for the 8 proteins in Table 2 (LBP, CSRP1, SERPINF2, PON1, RBP4, CST3, SERPINA4 and CFD), the potential of the biomarkers to distinguish the NOMOA, TCMR and BKVN groups and the ABMR group was evaluated and shown in [FIG. 6]. As a result, it was confirmed that the area under the curve was 0.5 or more in all of the biomarkers, and it showed a very high level in sensitivity and specificity.

4-5. Verification of ABMR-Specific Biomarkers

Among the 8 biomarkers selected in Example 4-4, verification was performed on the two types of LBP and CST3 which were selected through an additional step.

As in Example 1, urine samples were collected 2 to 3 hours before the biopsy from patients and donors who underwent an indication biopsy within 5 years of kidney transplantation. In addition, urine samples were collected from living kidney donors immediately before kidney donation surgery. Patients were classified into 5 groups according to the pathological diagnosis of biopsy: an antibody-mediated rejection (ABMR) group, a T cell-mediated rejection (ABMR) group, a BK virus nephropathy (BKVN) group, a group without major abnormalities (NOMOA) and a donor group (DONOR). Urine from patients with mixed allopathic lesions was excluded. This study was conducted with the approval of the Medical Institution Evaluation Committee at Man Medical Center, and written consent was obtained from all patients.

The protein expression levels of the two ABMR-specific markers were measured by western blotting for exosomes isolated from each urine sample from 11 donors corresponding to kidney donors (control), a group of 7 patients without major abnormalities (NOMOA) and an antibody-mediated rejection group (ABMR) of 10 patients. Information on the antibodies used for western blotting is shown in Table 3.

TABLE 3

| Antibody | Manufacturer | Cata. No |
|---|---|---|
| Human LBP antibody | R&D systems | AF870 |
| Human cystatin C (CST3) antibody | R&D systems | AF1196 |
| Anti-goat IgG + HPR | GenDeoot | SA007 |

In the group of patients with high severity of antibody-mediated rejection, proteins which were mixed from two patients representing the average was used as a positive control (PC) group. The intensity of the baud was numerically quantified using Image J software, and the result was analyzed by quantifying the result values of the ABMR group compared to the result values of the patients in the NOMOA group.

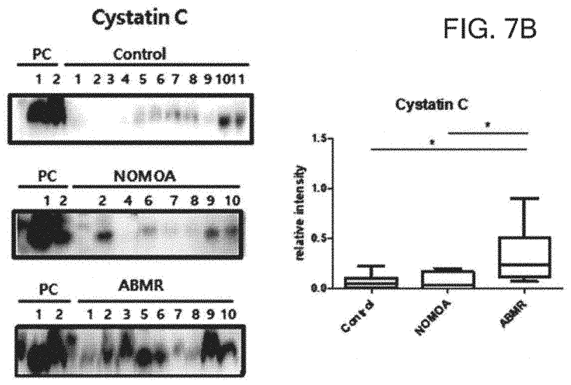

As can be seen in FIGS. 7a and 7b, LBP increased by 6.4 times in the ABMR group compared to the NOMOA group, and 10.7 times in the ABMR group compared to the normal control group. Cystatin C (CST3) was found to increase by 4.0 times in the ABMR group compared to the NOMOA group and 4.5 times in the ABMR group compared to the normal control group.

Accordingly, LBP and cystatin C (CST3) proteins were finally selected as the ABMR-specific markers whose statistical significance was verified, and as a result of obtaining the area under the ROC curve (AUC) values for the two markers, it was confirmed that LBP was 0.87 and cystatin C (CST3) was 0.84, all of which were statistically significant values. Therefore, through this Example, LBP and cystatin C (CST3) proteins were specifically highly expressed in ABMR patients, and thus, their values as ABMR-responsive biomarkers were sufficiently demonstrated.

The national research and development project that supported the present invention is as follows.

[National R&D Project That Supported This Invention]

[Task Identification Number] 1711046930/2016M3A9E8941330

[Ministry Name] Ministry of Science and Technology Information and Communication

[Research Management Professional Institution] Korea Research Foundation

[Research Project Name] Biomedical Technology Development Project

[Research Title] Development of urinary transglutaminase 2 diagnostic kit as a biomarker for fibrosis after kidney transplantation

[Host Institution] Seoul Asan Medical Center

[Research Period] Feb. 1, 2020 to Jul. 31, 2020

1-4. (canceled)

5: A kit for diagnosing antibody-mediated rejection after kidney transplantation or predicting the prognosis of a patient after kidney transplantation, comprising the composition of a material for measuring the expression level of any one or more proteins selected from the group consisting of LBP and CST3, or mRNA of a gene encoding the protein.

6-8. (canceled)

9. A method for providing information required for determining a therapy for rejection after kidney transplantation, comprising measuring the expression level of any one or more proteins selected from the group consisting of LBP and CST3, or mRNA of a gene encoding the same in a sample isolated from a subject.

10. The method of claim 9, wherein the sample is urine or exosomes derived from urine.

11. The method of claim 9, wherein the method further comprises determining as antibody-mediated rejection and determining to apply a therapy for antibody-mediated rejection, if the result of comparing the measured protein or mRNA expression level with a sample of a normal control group that did not receive kidney transplantation or a sample of a patient group that did not show rejection or BK virus nephropathy after kidney transplantation corresponds to any one or more selected from the group consisting of a) and b) below:
a) upregulation of LBP; and
b) upregulation of CST3.

12. A method for diagnosing and treating antibody-mediated rejection after kidney transplantation, comprising:
a) measuring the expression level of any one or more proteins selected from the group consisting of LBP and CST3, or mRNA of a gene encoding the same in a sample isolated from a subject;
b) comparing the expression level measured in step a) with a sample of a normal control group that did not receive kidney transplantation or a sample of a patient group that did not show rejection or BK virus nephropathy after kidney transplantation;
c) determining as antibody-mediated rejection after kidney transplantation, if, as a result of the comparison of step b), the expression level in the sample isolated from the subject of step a) is higher than the expression level of the sample of a normal control group that did not receive kidney transplantation or the sample of a patient group that did not show rejection or BK virus nephropathy after kidney transplantation; and
d) applying a therapy for antibody-mediated rejection to the subject of step a).

13. The method of claim 12, wherein the sample is urine or exosomes derived from urine.

14. The method of claim 12, wherein the therapy for antibody-mediated rejection of step d) is any one or more selected from the group consisting of steroid, plasmapheresis (PP), intravenous immunoglobulin (WIG), an anti-CD20 antibody (rituximab), a lymphocyte-depleting antibody, a proteasome inhibitor, a Cl-inhibitor and a monoclonal antibody against complement factor 5.

15. A method for screening a therapeutic agent for antibody-mediated rejection after kidney transplantation, comprising:
a) treating a candidate substance of a therapeutic agent for antibody-mediated rejection after kidney transplantation to a sample isolated from a subject exhibiting antibody-mediated rejection after kidney transplantation; and
b) measuring the expression level of any one or more proteins selected from the group consisting of LBP and CST3, or mRNA of a gene encoding the same.

16. The method of claim 15, wherein the sample is urine or exosomes derived from urine.

17. The method of claim 15, further comprising determining the candidate substance of step a) as a therapeutic agent for antibody-mediated rejection after kidney transplantation, if the expression level of any one or more proteins selected from the group consisting of LBP and CST3, or mRNA of a gene encoding the same, which was measured in step b), is downregulated compared to before treating the candidate substance.
